(19) United States
(12) Patent Application Publication
(10) Pub. No.: US 2026/0091217 A1
(43) Pub. Date: Apr. 2, 2026

(54) CABLE CONNECTOR WITH MEDICAL DEVICE CONSOLE MEMORY

(71) Applicant: BOSTON SCIENTIFIC SCIMED, INC., Maple Grove, MN (US)

(72) Inventors: Noe Mendivil Gutierrez, Maple Grove, MN (US); Corydon Carlson, Stillwater, MN (US); Heather Drury, Dripping Springs, TX (US); Judith Tiferes Wang, Shoreview, MN (US)

(73) Assignee: BOSTON SCIENTIFIC SCIMED, INC., Maple Grove, MN (US)

(21) Appl. No.: 19/341,214

(22) Filed: Sep. 26, 2025

Related U.S. Application Data
(60) Provisional application No. 63/699,888, filed on Sep. 27, 2024.

(51) Int. Cl. A61M 60/585; A61M 60/13; A61M 60/216; A61M 60/508; G16H 40/63

(52) U.S. Cl. CPC A61M 60/585; A61M 60/13; A61M 60/216; A61M 60/508; G16H 40/63; A61M 2205/3569; A61M 2205/52

(57) ABSTRACT

A cable connector device, such as a single-connector cable device, and associated methods are disclosed herein. Example devices and methods may include a connector for connection to a medical device console. The connector may be disposed proximate an end of a cable of the cable connector device. The connector may include a housing and a connection interface adapted to couple the connector to the medical device console. A memory storage element may be disposed within the housing of the connector, and include a firmware implementation element adapted to record data and events from a previous medical device console to the memory storage element to store data and events within the memory storage element from a previous medical device console. The memory storage element may be adapted to transmit the stored data and events to a subsequent medical device console upon coupling the connector to the subsequent medical device console.

### CROSS REFERENCE TO RELATED APPLICATIONS

The present application claims the benefit of U.S. Provisional Patent Application Ser. No. 63/699,888, filed Sep. 27, 2024, the disclosure of which is incorporated herein by reference.

### TECHNICAL FIELD

The present disclosure relates to the field of medical devices and associated medical systems. More particularly, the present disclosure relates to the transfer of data and information from one medical device and/or system to another medical device and/or system.

### BACKGROUND

Rapid exchange of data on data-reporting consoles may be required to further assist a patient or subject, especially during instances when a patient or subject must be transported to an emergency hospital setting. For instance, it is often the case that a medical device console (such as a blood pump console as will be further described herein) controlling operation of a medical device (such as a percutaneous blood pump) does not contain fundamental data vital for operation of the medical device in use within the patient if previous data associated with operation of the medical device is not transferred from a previous medical device console that was previously connected to the medical device while in use in the patient or subject. In some instances, the initial medical device console that the medical device was previously connected to may need to be exchanged for another medical device console to continue operation of the medical device within the patient, such as in instances, in which the patient is transported to a hospital, from a first hospital to another hospital, from a first area of a hospital to another area of the hospital, etc. As such, an unmet need exists in the art for a way to transmit stored data and events from an initially connected medical device console to a subsequent connected medical device console.

### BRIEF SUMMARY

This disclosure provides design, material, manufacturing method, and use alternatives for cable connectors, such as cable connectors for medical devices and other related devices.

An example is a cable connector device for connection to a medical device console for controlling a medical device. The cable connector device of this and other examples may include a cable having a first end, a second end, and a length extending therebetween. The cable connector device of this and other examples may include a connector disposed proximate the cable second end, the connector may further include a housing and a connection interface adapted to couple the connector to a medical device console. The cable connector device of this and other examples may further include a memory storage element disposed within the housing, the memory storage element is adapted to store data and events from a previous medical device console previously coupled to the connector, and transmit the stored data and events to a subsequent medical device console upon coupling the connector to the subsequent medical device console.

Alternatively or additionally to any of the examples disclosed herein, the memory storage element may include one or more of an EEPROM chip, a firmware implementation element, and a writing protection scheme.

Alternatively or additionally to any of the examples disclosed herein, the stored data and events may include one or more of: motor sensor coefficients, pressure sensor coefficients, case settings, list of case clinical events, configuration settings, diagnostics information, alarm information, pump speed changes, pump position, motor speed changes, notifications, and key values.

Alternatively or additionally to any of the examples disclosed herein, the connector includes one or more electrical connections and one or more sensor connections.

Alternatively or additionally to any of the examples disclosed herein, the memory storage element may include a writing protection scheme to prevent data corruption during normal operation or system shutdown of a connected medical device console.

Alternatively or additionally to any of the examples disclosed herein, the connector may be a right-angle round connector.

Alternatively or additionally to any of the examples disclosed herein, the first end of the cable is connected to a medical device, preferably a percutaneous blood pump.

Alternatively or additionally to any of the examples disclosed herein, the memory storage element may include a firmware implementation element configured to record data and events onto the memory storage element.

Alternatively or additionally to any of the examples disclosed herein, the subsequent medical device console saves the transmitted stored data and events upon coupling with the connector and rewrites the stored data and events within the memory storage element in the event of a warning or alert issuing from the subsequent medical device console.

Methods are further contemplated and may incorporate any and all examples described and/or alluded to herein. In some examples, methods for rapidly exchanging data between medical device consoles in use with treating a patient or subject are disclosed. A method for rapidly exchanging data between medical device consoles may include receiving data associated with a patient or subject connected to a first medical device console via a cable. The cable may have a first end, a second end, a length extending therebetween, and a connector disposed proximate the second end. The connector may include a memory storage element adapted to store data and events from the first medical device console. The methods of this and other examples may further include: disconnecting the cable via the connector from the first medical device console; connecting the cable via the connector to a second medical device console; and transferring the stored data and events from the memory storage element to the second medical device console.

Alternatively or additionally to any of the examples and/or methods disclosed herein, the stored data and events may include one or more of: motor sensor coefficients, pressure sensor coefficients, case settings, list of case clinical events, configuration settings, diagnostics information, alarm information, speed changes, position changes, motor speed changes, notifications, and key values.

Alternatively or additionally to any of the examples and/or methods disclosed herein, the memory storage unit may include a writing protection scheme to prevent data corruption during normal operation or system shutdown of a connected medical device console.

Alternatively or additionally to any of the examples and/or methods disclosed herein, the step of connecting the cable via the connector to a second medical device console may further include: transferring a history of clinical events from the first medical device console to the second medical device console.

Alternatively or additionally to any of the examples and/or methods disclosed herein, methods disclosed may further include the step of saving the transmitted stored data and events to the second medical device console and rewriting the stored data and events within the memory storage element in the event of a warning or alert issuing from the second medical device console.

Alternatively or additionally to any of the examples and/or methods disclosed herein, methods disclosed may further include recording data and events from the first medical device console onto the memory storage element with a firmware implementation element.

Alternatively or additionally to any of the examples disclosed herein, a percutaneous cardiac assist device may include a blood pump configured to pump blood, a cable having a first end extending from the blood pump, a second end, and a length extending therebetween. A connector may be disposed proximate the cable second end, the connector including a housing and a connection interface configured to couple the connector to a pump console. The connector may further include a memory storage element disposed within the housing. The memory storage element may include a firmware implementation element configured to record data and events from a previous pump console to the memory storage element to store data and events within the memory storage element from a previous pump console. The memory storage element may be adapted to transmit the stored data and events to a subsequent pump console upon coupling the connector to the subsequent pump console.

Alternatively or additionally to any of the examples and/or methods disclosed herein, the connector may include a connector interface having one or more electrical connections and one or more sensor connections.

Alternatively or additionally to any of the examples and/or methods disclosed herein, the memory storage element further includes one or more of an EEPROM chip and a writing protection scheme.

Alternatively or additionally to any of the examples and/or methods disclosed herein, Alternatively or additionally to any of the examples and/or methods disclosed herein, the stored data and events include one or more of: motor sensor coefficients, pressure sensor coefficients, case settings, list of case clinical events, configuration settings, diagnostics information, alarm information, pump speed changes, pump position, motor speed changes, notifications, and key values.

Alternatively or additionally to any of the examples and/or methods disclosed herein, the memory storage element includes a writing protection scheme to prevent data corruption during normal operation or system shutdown of a connected pump console.

Alternatively or additionally to any of the examples and/or methods disclosed herein, the subsequent console saves the transmitted stored data and events upon coupling with the connector and rewrites the stored data and events within the memory storage element in the event of an alarm or alert issuing from the subsequent pump console.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense.

### BRIEF DESCRIPTION OF THE SEVERAL VIEWS OF THE DRAWINGS

The drawings illustrate the design and utility of preferred embodiments of the present disclosure. It should be noted that the figures are not drawn to scale and that elements of similar structures or functions are represented by like reference numerals throughout the figures. In order to better appreciate how the above-recited and other advantages and objects of the present disclosure are obtained, a more particular description of the present disclosure briefly described above will be rendered by reference to specific embodiments thereof, which are illustrated in the accompanying drawings. Understanding that these drawings depict only typical embodiments of the invention and are not therefore to be considered limiting of its scope, the present disclosure will be described and explained with additional specificity and detail through the use of the accompanying drawings, in which:

FIG. 1 illustrates an example blood pump, connectable to a blood pump console, in accordance with the present disclosure.

FIG. 2 illustrates a patient being transported while remaining in connection with a data-reporting console via a cable and a connector.

FIG. 3A illustrates a patient in a hospital and/or clinical setting, attached to a data-reporting console via a cable and a connector.

FIG. 3B illustrates a rapid exchange of consoles in which a cable and/or connector is disconnected from a previous console and connected to a subsequent console.

FIG. 4 illustrates an example connector in accordance with the present disclosure.

FIG. 5 illustrates an example data-reporting console in accordance with the present disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5). As used in this disclosure and appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc. Additionally, the term “substantially” when used in reference to two dimensions being “substantially the same” shall generally refer to a difference of less than or equal to 5%.

It is noted that references in the specification to “an embodiment”, “some examples”, “other examples”, etc., indicate that the embodiment described may include one or more particular features, structures, and/or characteristics. However, such recitations do not necessarily mean that all examples include the particular features, structures, and/or characteristics. Additionally, when particular features, structures, and/or characteristics are described in connection with one embodiment, it should be understood that such features, structures, and/or characteristics may also be used connection with other examples whether or not explicitly described unless clearly stated to the contrary.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following detailed description should be read with reference to the drawings in which similar elements in different drawings are numbered the same. The drawings, which are not necessarily to scale, depict illustrative examples and are not intended to limit the scope of the disclosure.

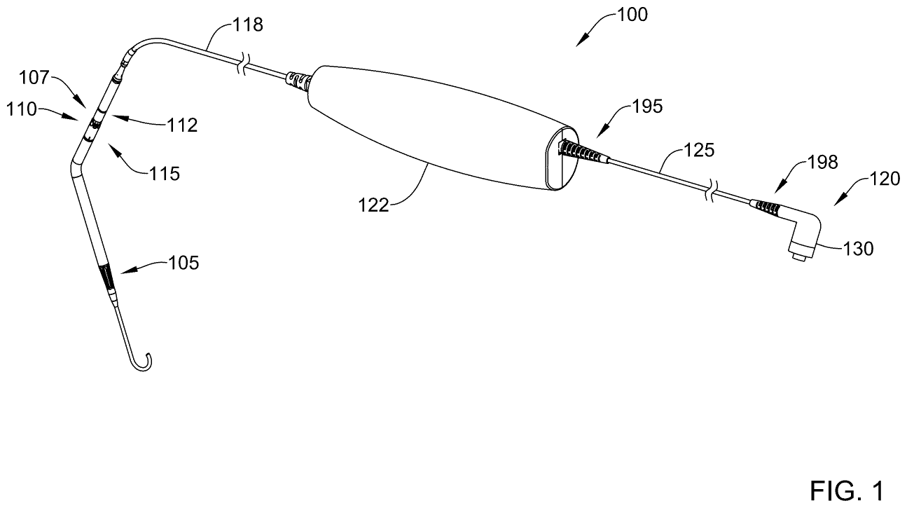

FIG. 1 shows an example percutaneous blood pump 100 (i.e., percutaneous cardiac assist device and/or circulatory support device) for connection to a medical device console (not shown). In the cases of blood pumps such as that depicted in FIG. 1, percutaneous cardiac assist devices and/or circulatory support devices support the pumping action of the heart. These devices may be positioned percutaneously into a patient's heart, such as disposed across a valve opening such as, for example, an aortic valve. Such percutaneous blood pumps 100 assist the heart in pumping blood throughout the cardiovascular system, and the present disclosure provides uses, devices, and methods incorporating such devices as will be described herein. Such percutaneous blood pumps 100 may be connected to a medical device console for operating the blood pump and/or monitoring the operation of the blood pump and/or one or more patient parameters. However, a wide variety of medical devices are utilized to treat patents which are connected to a medical device console for operating the medical device and/or monitoring the operation of the medical device and/or one or more patient parameters. Accordingly, aspects of the present disclosure may be incorporated into a wide variety of medical devices.

Typically, a circulatory support device such as the example blood pump 100 shown in FIG. 1 includes a blood pump housing 115 including a blood inlet 105 at a blood inflow end of the blood pump housing 115 and a blood outlet 107 at a blood outflow end of the blood pump housing 115. The blood pump housing 115 may include an impeller 110 or other blood pumping device configured to convey blood through the blood pump housing 115 from the blood inlet 105 to the blood outlet 107. In some instances, the impeller 110 may be disposed within an impeller housing portion of the blood pump housing 115. The impeller 110 may be constructed of radially oriented blades protruding from a drive shaft and/or drive mechanism, for example. In some instances, the blood inlet 105 may be positioned and/or be positionable in a ventricle of the patient, while the blood outlet 107 may be positioned and/or be positionable in the aorta of the patient, such that the blood pump 100 pumps blood across the aortic valve. The impeller 110 may be driven by a motor 112 or other means of rotating the impeller 110 during operation thereof. In some instances, the motor 112 may be housed within a motor housing portion of the blood pump housing 115. In other instances, the motor 112 may be arranged at another location, if desired, for example, the motor 112 may be located on the blood pump 100 such that it remains outside the body when the blood pump 100 is in use. The motor 112 or other means of rotating the impeller 110 may be configured to rotate the impeller 110 through magnetism, electromagnetism, mechanical force, electrical force, and/or any combination or permutation of the aforementioned, such that the drive shaft and/or drive mechanism rotates the blades of the impeller 110, thereby creating a blood flow to propel blood through the blood pump housing 115 and beyond into the cardiovascular system. The blood pump 100 may include an elongate catheter shaft 118 extending proximally from the blood pump housing 115 for navigating the blood pump 110 to a desired location within the cardiovascular system of the patient. The catheter shaft 118 may extend proximally to a junction box 122, a handle, a manifold, or other structure of the blood pump 100 configured to remain exterior of the patient.

Furthermore, the blood pump 100 may include a cable 125 at a proximal end thereof having a connection interface 120 (e.g., connector) at an end thereof. The cable 125 may have a first, distal end 195 connected to the junction box 122, a handle, a manifold, or other structure of the blood pump 100, and a second, proximal end 198 having the connection interface 120 including a connector 130 that is configured to be connected to a medical device console (e.g., blood pump console). The cable 125 may include one or more of, an electrical pathway for powering the motor 112, an electrical pathway for powering one or more sensors, an electrical pathway for transmitting signals from one or more sensors associated with the blood pump 100, a fiber optic filament for transmitting signals from one or more sensors associated with the blood pump 100, or other communication conduits. In some instances, the cable 125 and associated connector 130, may be considered a single-connector cable device providing the only connection between the blood pump 100 and a medical device console controlling the blood pump 100.

Various other medical devices in lieu of a blood pump 100 and/or other like circulatory devices are contemplated by the present disclosure and will be described further herein. For example, other circulatory support devices may include alternative blood pumping structures for conveying blood through a patient's cardiovascular system from a blood inlet of the medical device to a blood outlet of the medical device. The circulatory support device may include or be couplable to a medical device console via the cable 125 and associated connector 130 positioned at the end of the cable 125. Similar to that described above, the cable 125 may include one or more of, an electrical pathway for powering the an electrical device of the circulatory support device, an electrical pathway for powering one or more sensors, an electrical pathway for transmitting signals from one or more sensors associated with the circulatory support device, a fiber optic filament for transmitting signals from one or more sensors associated with the circulatory support device, or other communication conduits. Furthermore, other medical devices including or couplable to a medical device console via the cable 125 and associated connector 130 positioned at the end of the cable 125 are contemplated. Moreover, the cable 125 may include one or more of, an electrical pathway for powering an electrical device of the medical device, an electrical pathway for powering one or more sensors, an electrical pathway for transmitting signals from one or more sensors associated with the medical device, a fiber optic filament for transmitting signals from one or more sensors associated with the medical device, or other communication conduits.

Although aspects of the cable connector device, including the cable 125 and associated connector 130, are further described herein in association with connection of the percutaneous blood pump 100 with a blood pump console 135, 155, it is understood that disclosure is amenable to the inclusion of the cable connector device with another medical device for connecting the medial device to an associated medical device console operating the medical device.

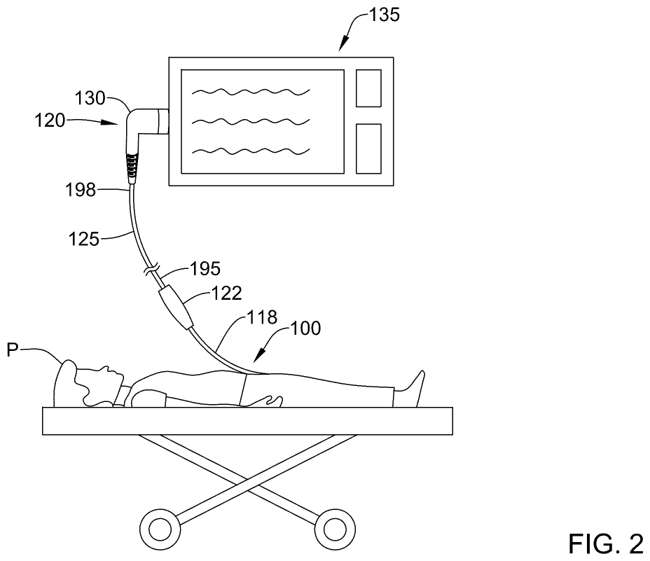

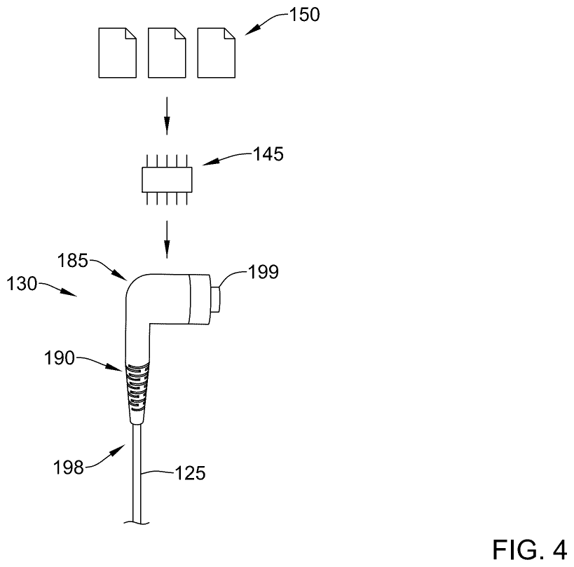

FIG. 2 shows an example cable connector device, such as a single-connector cable device, of the present disclosure connecting the blood pump 100 to a medical device console 135 (e.g., blood pump console) via a connection interface 120 in an ambulatory and/or emergency setting, such as during transport to a medical clinic and/or hospital. For example, the connector 130 may be directly connected to a connector of the medical device console 135 to provide electrical communication therebetween. The medical device console 135 may be configured to operate the blood pump 100 (or other medical device) and/or monitor the operation of the blood pump 100 (or other medical device) and/or one or more patient parameters. It is noted that the cable connector device may alternatively be used to connect another medical device, connected to a patient or subject (P), to a medical device console, in an ambulatory and/or emergency setting, such as during transport to a medical clinic and/or hospital. The cable 125 of this and other examples may couple or otherwise connect to a medical device console 135, such as a blood pump console and/or other data-reporting consoles that receive and/or display data from a blood pump and/or other medical device, through a connector such as connector 130 shown in FIG. 2 and in expanded detail in FIG. 4.

A first end 195 of the cable 125 may be connected to the blood pump 100 (or other medical device), while the connector 130 is located at a second end 198 of the cable 125 for coupling to the medical device console 135. A majority of the blood pump 100 may be inserted into the cardiovascular system of the patient through an access site, with only a proximal end portion of the catheter shaft 118 located external of the patient. The connector 130 may include a housing or enclosure. Within the housing or enclosure, one or more memory storage elements 145 (shown in FIG. 4) may be disposed, as will be described further herein. The provision of the memory storage element 145 allows the connector 130 to store data and events recorded by the console 135 onto the memory storage element 145, and further allows the connector 130, via the memory storage element 145, to instantaneously transmit, download, upload, or otherwise transfer the stored data and events recorded by the console 135 to a subsequent console immediately upon connection of the subsequent console to the connector 130, as will be further described herein. In the ambulatory and/or transport situation shown in FIG. 2, the console 135 may be transported with the patient from a first location to a second location, such as from a first hospital to a second hospital, from a first hospital room to a second hospital room, etc., while the blood pump 100 remains inserted into the patient's cardiovascular system and operating via the console 135.

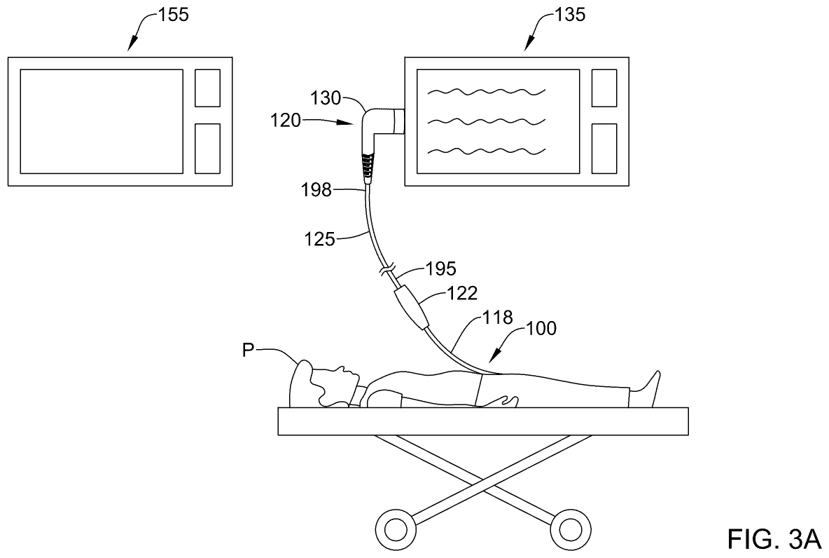

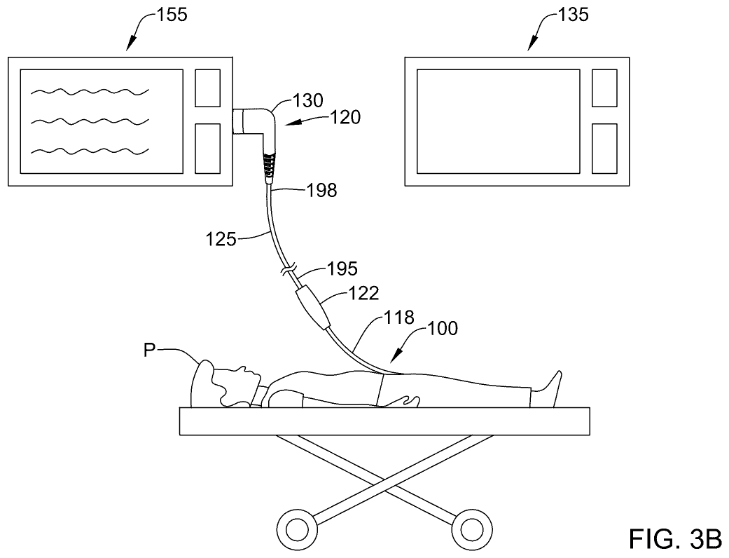

FIG. 3A shows an example situation in which the patient has been transferred to a second location, such as a hospital bed in another hospital or another hospitable bed in the hospital, etc., while the blood pump 100 is still connected to the console 135 via the connector 130.

Once transported to the second location, the cable 125 of the blood pump 100 may be disconnected from the console 135 (i.e., first console) and reconnected to another console 155 (i.e., second or subsequent console). FIG. 3B shows the cable connector device of the present disclosure as it is connected to a subsequent console 155 via the connector 130. In other words, FIG. 3B shows the connector 130 coupled or otherwise connected to the subsequent console 155 at a time after disconnection from the previous console, console 135. In some instances, the connector 130 may be switched or disconnected from one console and connected to another, subsequent console rapidly such that procedural time can be reduced for a patient and/or subject, and stored data and events from a previous console (such as console 135) may be instantaneously transferred to a subsequent console (such as subsequent console 155). It can be appreciated that this rapid switching of the connector 130 from a previous console 135 to a subsequent console 155 will not be hindered by the operating preferences, systems, and/or settings of the subsequent console. In other words, and as will be described further herein, the memory storage element 145 of the connector 130 is adapted to transmit stored data and events from a previous console 135 to a subsequent console 155, regardless of the compatibility of either console (135, 155). This is due in part because the memory storage element 145 may be adapted and/or otherwise configured to include and/or incorporate EEPROM (electronically erasable programmable read-only memory), Flash memory, and/or other like elements that will be described further herein.

In use with another medical device, the console 135 may be transported with the patient from a first location to a second location, such as from a first hospital to a second hospital, from a first hospital room to a second hospital room, etc., while the medical device remains connected to the patient and operating via the console 135. The patient may be transferred to a second location, such as a hospital bed in another hospital or another hospitable bed in the hospital, etc., while the medical device is still connected to the patient and operated by the console 135 via the connector 130.

Once transported to the second location, the cable 125 of the medical device may be disconnected from the console 135 (i.e., first console) and reconnected to another console 155 (i.e., second or subsequent console) via the connector 130. In other words, the connector 130 may be coupled or otherwise connected to the subsequent console 155 at a time after disconnection from the previous console, console 135. In some instances, the connector 130 may be switched or disconnected from one console and connected to another, subsequent console rapidly such that procedural time can be reduced for a patient and/or subject, and stored data and events from a previous console (such as console 135) may be instantaneously transferred to a subsequent console (such as subsequent console 155). It can be appreciated that this rapid switching of the connector 130 from a previous console 135 to a subsequent console 155 will not be hindered by the operating preferences, systems, and/or settings of the subsequent console. In other words, and as will be described further herein, the memory storage element 145 of the connector 130 is adapted to transmit stored data and events from the previous console 135 to the subsequent console 155, regardless of the compatibility of either console (135, 155).

FIG. 4 shows details of an example connector 130 of the present disclosure. The connector 130 of this and other examples may include a housing or enclosure which may be adapted to house or contain one or more memory storage elements 145. The connector 130 of this and other examples may be a right-angle round connector, as is depicted in FIG. 4. However, other shapes, geometries and configurations are also contemplated.

As shown in FIG. 4, and applicable to any and all examples disclosed herein, the housing of the connector 130 may include a connector body 185 and a connector stem 190. Connector body 185 may be unitary (i.e., of one-piece construction), or may be composed and/or comprised of multiple parts and/or portions and/or multiple materials. Connector body 185 may include a housing configured to house memory storage element 145 within the bounds of the connector body 185.

The connector stem 190 may be connected to the cable 125. As shown in FIG. 4, and applicable to any and all examples disclosed herein, connector stem 190 may extend from the connector body 185 at a right-angled connection (i.e., at a 90-degree angle with respect to the length of the connector body as shown in FIG. 4). In some instances, the connector stem 190 may extend generally coaxial with the connector body 185. In this and other examples, the connector stem 190 may be coupled with and/or otherwise be connected to connector body 185 at an angle less than 90 degrees, an angle less than 60 degrees, an angle less than 30 degrees, an angle more than 90 degrees, an angle more than 120 degrees, and/or an angle more than 150 degrees with respect to the length of the connector body 185. Alternatively or additionally, connector stem 190 may be molded to the connector body 185, and/or may be formed as a unitary structure with the connector body 185.

Further shown in FIG. 4 is connection interface 199 of the connector 130. Connection interface 199 may be disposed proximate the connector body 185 and utilized for connection with any medical device console disclosed herein and/or related medical device feasible for connection. Connection interface 199 may take the form of a male-female coupling, a female coupling member, a male coupling member, a threaded member, an internally threaded member, an externally threaded member, an interference-fit connection, a snap-fit connection, an electronic connection, a series of terminals, a conductor, or any like connection and/or coupling known in the art. The connection interface 199 may connect one or more electrical components and/or sensors of the blood pump 100 to the console connected thereto.

Alternatively or additionally to the examples disclosed herein, memory storage element 145 may include a firmware implementation element (not shown) that is adapted to and/or otherwise configured to record data and events 150 from a previous medical device console (such as console 135) to the memory storage element 145 to store data and events 150 within the memory storage element 145 from a previous medical device console (such as console 135).

In some instances, the memory storage element 145 may be configured to store data associated with the current settings for operating the blood pump (or other medical device) with the currently connected console (such as console 135), for transferring the current settings for operating the blood pump (or other medical device) to the subsequently connected console (such as console 155). For example, the current speed of the blood pump and/or current input electrical current to a motor running the blood pump may be transferred from the console the connector 130 is being disconnected from to the console the connector 130 is subsequently being connected to continue operation of the blood pump. In other instances, other operating parameters, past events, alerts, etc. associated with the medical device may be transferred from the console the connector 130 is being disconnected from to the console the connector 130 is subsequently being connected to continue operation of the medical device.

In some instances, the memory storage element 145 may be configured to store a plurality of past events, such as 20 or more past events, 30 or more past events 50 or more past events, 75 or more past events, or 100 or more past events. The firmware implementation element confers rewrite ability to the memory storage element 145, by utilizing firmware such as, but not limited to low-level firmware, high-level firmware, and/or subsystems firmware. Further, the firmware implementation element may be further adapted to and/or otherwise configured to read configuration settings from a console, a previous console connected to the connector 130 (such as console 135), and/or a subsequent console upon connection via the connector 130 (such as console 155). Alternatively or additionally, the firmware implementation element may be adapted to and/or otherwise configured to implement a writing protection scheme to prevent data corruption during normal operation and/or system shutdown of a connected console, and/or any connected medical and/or peripheral device associated with a blood pump, circulatory support system, or other medical device.

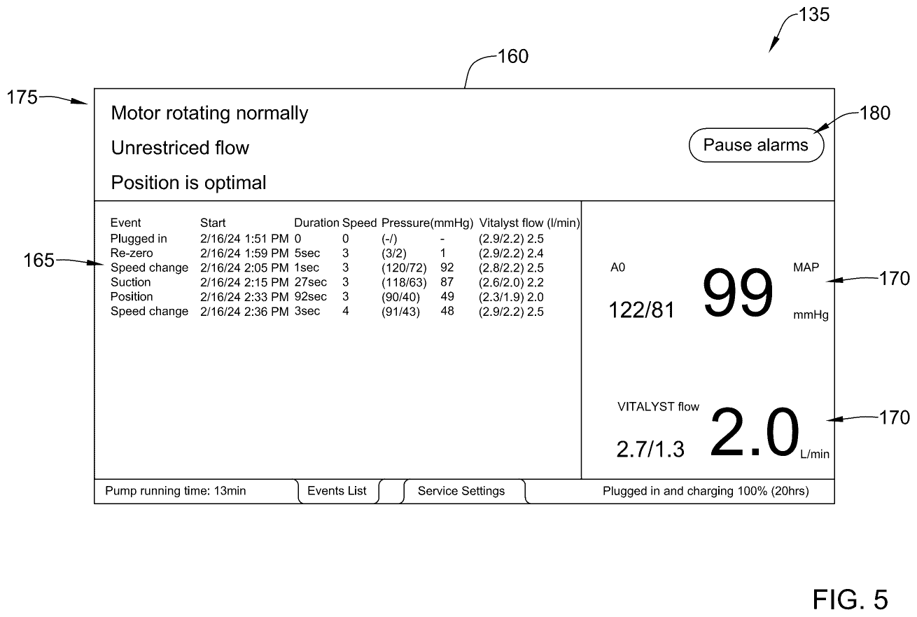

FIG. 5 shows an example console display 160 of a console 135 (or console 155) applicable to any of the examples disclosed herein. Console display 160 may include and/or display status indicators 175 (e.g., motor operation conditions, flow conditions, pump position conditions), displayed data and events 165, display outputs 170, and input interface 180.

Status indicators 175 may alternatively and/or additionally include status indicators for a pump and/or other related and connected devices. Status indicators 175 may include, but are not limited to: pump connectivity status, device connectivity status, motor rotation status, pump power status, device power status, circulatory support system power status, circulatory support system connectivity status, pump occlusion status, pump suction status, pump position status, device position status, battery level status, low battery level status, critically low battery level status, or any equivalent or suitable status indicator.

Displayed data and events 165 may include, but are not limited to motor and pressure sensor coefficients, case settings, clinical case settings, diagnostics information, previous pump malfunctions, previous motor malfunctions, previous device malfunctions, previous console operation status, previous console power status, previous device operation status, previous device power status, pump connectivity status, previous pump connectivity status, console connectivity status, previous console connectivity status, pump position status, previous pump position status, speed change of a pump, speed change of a motor, speed change of a previous pump, speed change of a previous motor, suction pressure within a device, suction pressure within a previous device, suction pressure within a pump, suction pressure within a previous pump, and may display a large number of events and/or receive a large number of events from the connector 130 via the memory storage element 145, the large number of events may range from one or more events to 1,000 or more events.

Alternatively or additionally to any of the examples disclosed herein, the history of events and/or the history of clinical events may be saved by the console (135/155) upon connection to the connector 130 and may be saved to the memory storage element 145 of the connector 130 instantaneously upon an alarm and/or alert condition generated by any connected console, including but not limited to consoles 135 and 155. Alternatively or additionally to any of the examples disclosed herein, the history of events and/or the history of clinical events may be saved by the console (135/155) upon connection to the connector 130 and may be saved to the memory storage element 145 of the connector 130 at any time during normal pump or medical device operation. Accordingly, and applicable to all examples disclosed herein, no patient personal information will be saved and/or written to the memory storage element 145 of the connector 130 throughout use of the devices disclosed herein.

Display outputs 170 may include data and/or events received from a blood pump (such as blood pump 100) and/or other medical device that are displayed on the console 135 as shown in FIG. 5. Display outputs 170 may include, but are not limited to: minimum arterial pressure, maximum arterial pressure, mean arterial pressure, minimum blood flow rate, maximum blood flow rate, mean blood flow rate, minimum blood flow rate through the blood pump, maximum blood flow rate through the blood pump, mean blood flow rate through the blood pump, estimated blood pressure, estimated blood pressure waveform, left ventricular pressure, minimum left ventricular pressure, maximum left ventricular pressure, mean left ventricular pressure, minimum arterial oxygenation, maximum arterial oxygenation, mean arterial oxygenation, minimum arterial pressure waveform, continuous arterial pressure waveform, maximum arterial pressure waveform, mean arterial pressure waveform, blood flow rate waveform, continuous blood flow rate waveform, minimum blood flow rate waveform, maximum blood flow rate waveform and mean blood flow rate waveform.

Input interface 180 may be any input interface known in the art adapted to display the request, option, and/or discharge of inputs to and/or from a console (such as console 135/155), and/or a blood pump (such as blood pump 100) and/or other medical device capable of transmitting events and data. Input interface may include any input interface including but not limited to: touch screen interfaces, haptic feedback interfaces, audio-interactive interfaces (i.e. Siri, Alexa), mechanical input interfaces, remote interfaces, internet-connected interfaces, and/or any combination and/or permutation of the aforementioned and/or the like.

Blood pumps and/or other circulatory support devices of the present disclosure may be mechanically driven, pneumatically driven, magnetically driven, electronically driven, electronically actuated, mechanically actuated, magnetically actuated, electromechanically actuated, piezoelectrically actuated, and/or any combination or permutation of the aforementioned and/or the like.

Alternatively or additionally, circulatory support systems contemplated by this disclosure may include any device capable of circulatory support that is compatible with a user interface. Example circulatory support systems include but are not limited to left ventricular assist devices (LVAD), right ventricular assist devices (RVAD), biventricular assist devices (BiVAD), intraaortic balloon pumps (IABP), heart pumps, impeller pumps, artificial hearts, total artificial hearts (TAH) or any of the like or equivalent. Alternatively or additionally, cardiac circulatory systems may also be incorporated by the present disclosure in like manner as a circulatory support device. Example cardiac circulatory support devices include, but are not limited to, ventricular assist devices (VADs), total artificial hearts (TAH), intra-aortic balloon pumps (IABP), and extracorporeal membrane oxygenation (ECMO). Example VADs include left ventricular assist devices (LVADs), right ventricular assist devices (RVADs), and biventricular assist devices (BiVADs). A further illustrative VAD is a percutaneous ventricular assist device (PVAD), which may be inserted into a ventricle (e.g., a left ventricle or a right ventricle) of a heart of a patient via delivery through a femoral artery or vein and/or other suitable vasculature to the ventricle. As a PVAD may be placed at a desired location of anatomy of a patient via percutaneous access and delivery, the PVAD may be used in emergency medicine, a catheter laboratory, and/or other surgical and/or non-surgical settings.

Methods are additionally contemplated herein and may be utilized with and applicable to any of the elements, components, and/or devices disclosed herein. An example method for rapidly exchanging data between medical device consoles with a patient or subject is disclosed and may include receiving data associated with a patient or subject (P) connected to a first medical device console 135 via a cable 125. The cable 125 having a first end 195, a second end 198, a length extending therebetween and a connector 130 disposed proximate the second end 198. The connector may include a memory storage element 145 adapted to or otherwise configured to store data and events 150 from the first medical device console 135. Methods of this and other examples may further include disconnecting the cable 125 via the connector 130 from the first medical device console 135, connecting the cable 125 via the connector 130 to a second medical device console 155, and transferring the stored data and events 150 from the memory storage element 145 to the second medical device console 155.

Alternatively or additionally, methods disclosed herein may further provide that the stored data and events 150 include one or more of motor sensor coefficients, pressure sensor coefficients, case settings, clinical case settings, list of case clinical events, configuration settings, diagnostics information, alarm information, pump speed changes, pump position, motor speed changes, notifications and/or key values.

Alternatively or additionally, methods disclosed herein may further provide that the memory storage element 145 includes a writing protection scheme to prevent data corruption during normal operation and/or system shutdown of a connected medical device console.

Alternatively or additionally, methods disclosed herein may further provide that the memory storage element 145 includes transferring a history of clinical events from a first medical device console to a second medical device console when the cable is coupled to the second medical device console upon being disconnected from the first medical device console.

Alternatively or additionally, methods disclosed herein may include saving transmitted stored data and events 150 to the second medical device console 155 and rewriting the stored data and events within the memory storage element 145 in the event of a warning or alert issuing from the second medical device console.

Alternatively or additionally, methods disclosed herein may include recording data and events 150 from the first medical device console 135 onto the memory storage element 145 with a firmware implementation element.

It should be noted and can be appreciated that some of the FIGS. are schematic in nature and are not drawn to scale. Certain features are shown larger than their scale and certain features are omitted from some views for case of illustration.

It should also be noted that, as used in this specification and the appended claims, the singular forms include the plural unless the context clearly dictates otherwise. It should also be noted that the term “or” is generally employed in its sense including “and/or” unless the content clearly dictates otherwise.

The present disclosure has been described with reference to various specific and exemplary embodiments. Those skilled in the art will understand that changes may be made in details, particularly in matters of shape, size, material and arrangement of parts. Accordingly, various modifications and changes may be made to the examples and the embodiments. Additional or fewer components may be used, depending on the condition that is being treated by the electrosurgical ablation device and other related devices and components disclosed herein. It should be understood that many variations and modifications may be made while remaining within the spirit and scope of the present disclosure. The specifications and drawings are, therefore, to be regarded in an illustrative rather than a restrictive sense.

What is claimed is:

1. A cable connector device for connection to a medical device console for controlling a medical device, the cable connector device comprising:
a cable having a first end, a second end, and a length extending therebetween;
a connector disposed proximate the cable second end, the connector including a housing and a connection interface configured to couple the connector to the medical device console;
a memory storage element disposed within the housing, the memory storage element configured to store data and events from a previous medical device console previously coupled to the connector, and transmit the stored data and events to a subsequent medical device console upon coupling the connector to the subsequent medical device console.

2. The device of claim 1, wherein the memory storage element includes one or more of an EEPROM chip, a firmware implementation element, and a writing protection scheme.

3. The device of claim 1, wherein the memory storage element includes a writing protection scheme to prevent data corruption during normal operation or system shutdown of a connected medical device console.

4. The device of claim 1, wherein the stored data and events include one or more of: motor sensor coefficients, pressure sensor coefficients, case settings, list of case clinical events, configuration settings, diagnostics information, alarm information, pump speed changes, pump position, motor speed changes, notifications, and key values.

5. The device of claim 1, wherein the connector includes one or more electrical connections and one or more sensor connections.

6. The device of claim 1, wherein the first end of the cable is connected to a medical device, preferably a percutaneous blood pump.

7. The device of claim 1, wherein the memory storage element includes a firmware implementation element configured to record data and events onto the memory storage element.

8. The device of claim 1, wherein the subsequent medical device console saves the transmitted stored data and events upon coupling with the connector and rewrites the stored data and events within the memory storage element in the event of a warning or alert issuing from the subsequent medical device console.

9. A method for rapidly exchanging data between medical device consoles in use with treating a patient or subject, the method comprising:
receiving data associated with a patient or subject connected to a first medical device console via a cable; the cable having a first end, a second end, a length extending therebetween, and a connector disposed proximate the second end; the connector including a memory storage element configured to store data and events from the first medical device console;
disconnecting the cable via the connector from the first medical device console;
thereafter, connecting the cable via the connector to a second medical device console; and
transferring the stored data and events from the memory storage element to the second medical device console.

10. The method of claim 9, wherein the stored data and events include one or more of: motor sensor coefficients, pressure sensor coefficients, case settings, list of case clinical events, configuration settings, diagnostics information, alarm information, pump speed changes, pump position changes, motor speed changes, notifications, and key values.

11. The method of claim 9, wherein the memory storage element includes a writing protection scheme to prevent data corruption during normal operation or system shutdown of a connected medical device console.

12. The method of claim 9, the step of connecting the cable via the connector to a second medical device console further comprising: transferring a history of clinical events from the first medical device console to the second medical device console.

13. The method of claim 9, the method further comprising:
saving the transmitted stored data and events to the second medical device console and rewriting the stored data and events within the memory storage element in the event of a warning or alert issuing from the second medical device console.

14. The method of claim 9, the method further comprising:
recording data and events from the first medical device console onto the memory storage element with a firmware implementation element.

15. A percutaneous cardiac assist device, the device comprising:
a blood pump configured to pump blood;
a cable having a first end extending from the blood pump, a second end, and a length extending therebetween;
a connector disposed proximate the cable second end, the connector including a housing and a connection interface configured to couple the connector to a pump console; and
a memory storage element disposed within the housing, the memory storage element including a firmware implementation element configured to record data and events from a previous pump console to the memory storage element to store data and events within the memory storage element from a previous pump console;
wherein the memory storage element is configured to transmit the stored data and events to a subsequent pump console upon coupling the connector to the subsequent pump console.

16. The device of claim 15, the memory storage element further including one or more of an EEPROM chip and a writing protection scheme.

17. The device of claim 15, wherein the stored data and events include one or more of: motor sensor coefficients, pressure sensor coefficients, case settings, list of case clinical events, configuration settings, diagnostics information, alarm information, pump speed changes, pump position, motor speed changes, notifications, and key values.

18. The device of claim 15, wherein the connector includes a connector interface having one or more electrical connections and one or more sensor connections.

19. The device of claim 15, wherein the memory storage element includes a writing protection scheme to prevent data corruption during normal operation or system shutdown of a connected pump console.

20. The device of claim 15, wherein the subsequent console saves the transmitted stored data and events upon coupling with the connector and rewrites the stored data and events within the memory storage element in the event of an alarm or alert issuing from the subsequent pump console.
